(19) United States
(12) Patent Application Publication
(10) Pub. No.: US 2009/0258072 A1
(43) Pub. Date: Oct. 15, 2009

(54) LARGE ULTRAVIOLET ATTENUATING PIGMENTS

(75) Inventors: David Schlossman, Short Hills, NJ (US); Pascal Delrieu, Somerset, NJ (US); Yun Shao, Belle Mead, NJ (US)

Correspondence Address: THOMPSON HINE L.L.P.;Intellectual Property Group, P.O. BOX 8801, DAYTON, OH, 45401-8801, US

(73) Assignee: Kobo Products, Inc., South Plainfield, NJ (US)

(21) Appl. No.: 12/420,983

(22) Filed: Apr. 9, 2009

Related U.S. Application Data
(60) Provisional application No. 61/044,274, filed on Apr. 11, 2008.

(30) Foreign Application Priority Data
Apr. 8, 2009 (US) PCT/US2009/039929

(51) Int. Cl. A61K 9/14; A61K 8/02; A61Q 17/04

(52) U.S. Cl. 424/489; 424/59

(57) ABSTRACT

Large particle sunscreen powders useful as ingredients in cosmetic compositions and in dispersions for incorporation into cosmetic compositions comprise a UV shielding agent in a matrix material. The macroparticle powders can be used in a wide range of cosmetic formulations, including sunscreens, eyeshadow, mascara, foundation, blusher, toner, lipstick and other compositions requiring ultraviolet protection.

### CROSS REFERENCE TO RELATED APPLICATION

This application claims the benefit of U.S. Provisional Patent Application No. 61/044,274, filed Apr. 11, 2008, the contents of which are hereby incorporated by reference.

### TECHNICAL FIELD

The present invention relates to ultraviolet attenuating pigments, methods for forming the same and compositions containing the inventive pigments.

### BACKGROUND OF THE INVENTION

Background light scattering pigments have long been used ultraviolet protection. For many years, continuing to the present, ointments comprising large particles of titanium dioxide have been applied to the skin, typically seen the form of a white smear on the nose and cheeks. While consumer perception appears to be that such visually obvious applications confer a high degree of protection from ultraviolet radiation, it has long been known that much smaller zinc oxide and titanium dioxide particles suspended in a medium of, for example, oil, will not only attenuate ultraviolet light but will also appear transparent and thus invisible. This is achieved because the particles have a diameter substantially smaller than the wavelength of visible light. However, there are concerns that the particles may penetrate the skin.

### SUMMARY OF THE INVENTION

The present invention relates to large powders that act as ultraviolet (UV) shielding agents. The macroparticle powder prepared in accordance with certain aspects of the present invention contains an insignificant number of particles less than 100 nm, but can still effectively attenuate ultraviolet radiation while being transparent in the visible light region and thus not visible.

Ultraviolet light is known to decompose many kinds of materials and cause damage to skin such as premature aging, wrinkles, etc. Therefore, many compositions and formulations contain materials to block or filter out the high-energy ultraviolet light. Accordingly, compositions such as cosmetics, sunscreens, etc. typically contain some kind of UV blocking or filtering agent to prevent the deleterious effects of ultraviolet light. Ultraviolet shielding agents can be either organic or inorganic.

Organic UV shielding agents typically prevent the transmission of ultraviolet light as a result of the specific absorption of ultraviolet radiation by the organic compound.

Inorganic, or more properly “optical”, UV filters usually are in the form of nano-size particles which results in giving them high transparency. Inorganic UV filters are particularly useful as sunscreen agents because of their physical and chemical stability. Furthermore, they are typically non-irritating.

Certain aspects of the present invention relate to macroparticle powders that, despite their large size, keep the optical properties of the nanoparticles, at least from the standpoint of transparency and UV protection, and even though the particles are dispersed in a matrix material. In accordance with one aspect of the invention the matrix is formed of a transparent gel material. In accordance with certain aspects of the present invention, fine macroparticle powders, comprising matrix material and optical sunscreen nano particulates, are obtained that facilitate formulation and provide good skin feel.

The present invention achieves this result by forming a macroparticle powder particles. The macroparticle comprises a UV shielding agent in a matrix material. The macroparticle powders of the present invention find use in cosmetic formulations, sunscreens, and other compositions requiring ultraviolet protection.

In accordance with certain aspects of the present invention, the macroparticle powders containing UV shielding agents effectively block ultraviolet light and yet exhibit transparency in the visible light region. In accordance with certain aspects of the present invention, the macroparticle powder does not have a significant number of particles with size under 100 nm. Therefore, it has limited potential to penetrate human skin.

In accordance with other aspects of the present invention, particles less than 100 nm in size are incorporated into a matrix material to provide a macroparticle powder with an average particle size of greater than 100 nm and an insignificant number of particles under 100 nm in size.

In accordance with another aspect of the present invention, a method of protecting human skin or human hair from ultraviolet radiation is provided. The method comprises treating skin or hair with an effective protecting concentration of a composition comprising the macroparticle powder set forth herein.

The present invention also provides a sunscreen-containing personal care composition for protecting human skin or human hair from ultraviolet radiation which comprises an effective protecting concentration of the macroparticle powder described herein in a suitable carrier.

### DETAILED DESCRIPTION OF THE INVENTION

The term “macroparticle powder” as used herein refers to the particles produced by dispersing UV shielding agents in a solid matrix material. In accordance with certain aspects of the present invention, the macroparticle powder particles have an average particle size of between about 0.2 μm to about 1000 μm, and preferably from about 1 μm to about 30 μm. In accordance with a particularly preferred embodiment of the invention, the particles have a size ranging from about 3000 μm to about 10,000 μm.

The invention contemplates a macroparticle powder comprising a matrix supporting a plurality of particles of an optical sunscreen particle, that is a sunscreen particle that, in a conventional sunscreen tends to be transparent to visible light, but at the same time tends to block ultraviolet light. These particles of optical sunscreen material include titanium dioxide and zinc oxide having particle sizes in the range of, for example 5-300 nm, more preferably in the range of 10-250 nm and most preferably in the range of 20-200 nm. In addition to inorganic sunscreen particles, such as zinc oxide or titanium dioxide, the invention contemplates the use of polymeric materials having the characteristics of zinc oxide or titanium dioxide, as well as combinations of inorganic sunscreens, and combinations of inorganic sunscreens with such polymeric materials.

In accordance with the present invention, the inventive macroparticles may typically be incorporated in sunscreen compositions including polymeric organic UV shielding agents, for example, a triazine, an oxanilide, a triazole, a vinyl group-containing amide, a cinnamic acid amide, or a sulfonated benzimidazole UV shielding agent or other class of substance known as UV shielding agents.

Particle size and type may be varied depending on the range where attenuation is desired (for example UVA and UVB) and depending on the material type. In accordance with a certain embodiment of the present invention, the organic UV shielding agent comprises a micronized version of 2,2′-Methylene-bis-{6-(2H-benzotriazole-2-yl)-4-(1,1,3,3-tetramethylbutyl)-phenol} which is available commercially under the name TINOSORB M from Ciba Specialty Chemicals. The INCI name for TINOSORB M is Methylene Bis-Benzotriazolyl Tetramethylbutylphenol (MBBT). TINOSORB M is a 50% aqueous dispersion of micronized MBBT having a particle size less than 200 nm. The organic UV shielding agents may have an average particle size of from about 5 nm to about 200 nm, more preferably from about 10 nm to about 100 nm. In accordance with a most preferred embodiment of the present invention the particle size of the organic particles incorporated in the matrix ranges from about 20 nm to about 90 nm.

Examples of organic UV shielding nanoparticle particulates that may be useful in the present invention include those described in U.S. Pat. No. 5,869,030 to Dumler et al. and U.S. Pat. No. 6,495,122 to Fankhauser et al., the contents of these patents are hereby incorporated by reference.

The inorganic UV shielding agents useful in the present invention are those typically used for shielding ultraviolet light. In accordance with certain embodiments of the present invention, the inorganic UV shielding agents are metal oxides and more specifically metal oxides selected from TiO₂, ZnO, zirconium oxide, cerium oxide and any metal oxides or other materials that can absorb and/or scatter UV light while maintaining an acceptable degree of transparency, and mixtures thereof. The metal oxide particles may have an average particle size of from about 1 nm to about 150 nm, more preferably from about 5 nm to about 100 nm. In accordance with the most preferred embodiment of the present invention, particle size of the particulate in the matrix ranges from about 10 nm to about 35 nm.

The UV shielding agent particles incorporated in the matrix can be uncoated or can be coated (for example with a metal oxide or hydroxide), and/or organic compounds such as, but not limited to, fatty acids, metal soap, silicones, silanes, etc. The UV shielding agent particles can be hydrophilic or hydrophobic.

The macroparticle powder as described herein may contain a single type of UV shielding agent or combination of UV shielding agents. Furthermore, the UV shielding agents in the macroparticle powder may also be combined with additional substances, such as, for example, photostabilizers, cosmetic oils and/or anti-oxidants.

The matrix material is one that is capable of forming, for example, a gel to entrap the particles of the UV shielding agent or a material exhibiting sufficient adhesion to bind the UV shielding agent particles without significantly interfering with the ultraviolet filtering ability of the UV shielding agent particles or the transparency of the composition in the visible light region. In accordance with a particular aspect of the present invention, the matrix material comprises agar. Cellulose can also be used.

The inventive macroparticle powder typically comprises the UV shielding agent and matrix material present at a ratio (by dry weight) of about 1:1 to about 7:10, more preferably from about 3:1 to about 7:1. In accordance with a most preferred embodiment of the present invention the ratio varies from 4:1 to about 6:1. Each macroparticle powder particle typically contains a plurality of UV shielding agent particles. It is also contemplated that various particle types and/or particles of various sizes may be combined in a single macroparticle powder particle.

Macroparticle powders may be formed by any method capable of producing the macroparticle powder particles at the appropriate size. The present invention is described in more detail by reference to spray drying to form the macroparticle powder. However, the present invention should not be considered limited to this process and other processes such as freeze drying, prilling, extrusion/spherization, emulsion/dispersion process and precipitation may also be used.

It is also contemplated that particle formation may be followed by screening, or other processes to assure proper particle size.

Spray drying is a particle processing technology that transforms a liquid feed stock into a powder product by first spraying the feed stock to create droplets, and then evaporating the feed stock liquid through the use of a heated drying medium, typically air. The liquid feed stock can take the form of a solution, suspension, liquid-paste or emulsion, and should be pumpable and capable of droplet formation. The feed stock composition in accordance with the present invention comprises the UV shielding agent, the matrix material and a dispersion media, such as, for example, water or organic solvents.

The UV shielding agent macroparticle powder prepared in accordance with the present invention may be formulated into cosmetic compositions, sunscreen compositions, or other compositions as needed to provide the desired ultraviolet filtering properties. The UV shielding agent macroparticle powder may be incorporated into the finished compositions with a concentration of UV shielding agent from about 1 to about 80% by weight, more preferably from about 2-20%, and most preferably from about 3 to about 10% by weight to provide an effective and typical protecting concentration of the ultraviolet shielding agent. The finished compositions may be in the form of suntan lotions, bronzers, other lotions, gels, hairsprays, mascara, foundation, face powder, aerosol foam creams or emulsions, and so forth.

The cosmetics of the present invention may be formulated in various forms by conventional methods. Although the forms are not particularly limited, the cosmetics may be formulated as various makeup products as noted above and including lotions, emulsions, creams, ointments, aerosol cosmetics, powdery foundations, powdery eye shadows, emulsifying foundation creams, lipsticks, hair care preparations, and skin cleansers.

Generally, the inventive macroparticles function, from an optical standpoint, in the same manner as the much smaller nanoparticles of the sunscreen, because the index of refraction of the matrix is substantially lower than the index of refraction of the sunscreen particles, whether the sunscreen particles be polymeric or metal oxides.

The present invention is described in more detail by the following non-limiting examples.

### Example 1

An agar solution was prepared by thoroughly mixing 0.4 kg of agar with 10 kg of tap water. Mixing was performed in a jacketed tank heated to 194° F. The result was an approximately 4% agar solution.

A particulate zinc oxide suspension was prepared by mixing 1.11 kg of Kobo WS55XZ4 with 8.49 kg of water. Kobo WS55XZ4 available from Kobo Products, Inc. and is a suspension of zinc oxide. The Kobo WS55XZ4 was mixed with the water and heated to 140° F. A high speed homogenizer may be used to break up clumps that form in the tank.

The agar solution and the ZnO suspension were then combined to make a sunscreen/agar mixture. The sunscreen/agar mixture was maintained at 155° F. and spray dried using a conventional spray drying apparatus.

Particle size of the spray dried product was first assessed under an optical microscope. The agar-ZnO powder included a large number of very small particles (1 to 5 μm) and some larger particles (20 μm) were observed. Particles did not appear to be all spherical. A further particle size measurement was made using a light scattering method, and a mean size (volume weighted) of 13 μm (60% dispersion) was measured.

Moisture of the powder collected from the vortex of the spray dry apparatus was measured at 2.8%

### Example 2

An agar solution was prepared by thoroughly mixing 0.4 kg of agar with 10 kg of tap water. Mixing was performed in a jacketed tank heated to 194° F. The result was an approximately 4% agar solution.

A particulate titanium dioxide suspension was prepared by mixing 1.33 kg of Kobo W45AQ with 8.49 kg of water. Kobo W45AQ is available from Kobo Products, Inc. and is a 45% aqueous dispersion of 15 nm TiO₂. The Kobo W45AQ was mixed with the water and heated to 140° F. A high speed homogenizer may be used to break up clumps that form in the tank.

The agar solution and the titanium dioxide suspension were then combined to make a sunscreen/agar mixture. The sunscreen/agar mixture was maintained at 140° F. and spray dried using a conventional spray drying apparatus.

### Example 3

An agar solution was prepared by thoroughly mixing 0.8 kg of agar with 20 kg of tap water. Mixing was performed in a jacketed tank heated to 194° F. The result was an approximately 4% agar solution.

A particulate polymeric sunscreen suspension was prepared by mixing 1.2 kg of TINOSORB M in 18 kg of water. TINOSORB M is available from Ciba Specialty Chemicals. The TINOSORB M was mixed with the water and heated to 194° F. while mixing. A high speed homogenizer may be used to break up clumps that form in the tank.

The agar solution and the sunscreen suspension were then combined to make a sunscreen/agar mixture. The sunscreen/agar mixture was maintained at 140° F. and spray dried using a conventional spray drying apparatus.

What is claimed is:

1. A macroparticle sunscreen powder comprising a plurality of macroparticle particles, said macroparticle particles comprising UV attenuating particles in a matrix material.

2. A macroparticle sunscreen powder in claim 1 wherein said matrix material has an index of refraction lower than the index of refraction of said UV attenuating particles.

3. A macroparticle sunscreen powder as in claim 2, wherein said matrix material is transparent or translucent.

4. A macroparticle powder as in claim 2, wherein, said macroparticle particles have an average particle size of from about 0.2 μm to about 1,000 μm, and said UV attenuating particles having an average size between 5 and 350 nm.

5. A macroparticle powder in accordance with claim 4 wherein the UV shielding agent particles have an average particle size of less than 200 nm.

6. A macroparticle powder in accordance with claim 5 wherein the UV shielding agent comprises inorganic UV attenuating particles.

7. A macroparticle powder in accordance with claim 6 wherein the inorganic UV filter comprises metal oxide particles selected from the group consisting of TiO₂, ZnO and combinations of TiO₂ and ZnO.

8. A macroparticle powder in accordance with claim 7 wherein said metal oxide particles have an average particle size of from about 5 nm to about 50 nm.

9. A macroparticle powder in accordance with claim 5 wherein the UV shielding agent comprises micronized organic UV attenuating particles.

10. A macroparticle powder in accordance with claim 2 wherein the UV shielding agent comprises micronized organic UV attenuating particles.

11. A macroparticle powder in accordance with claim 9 wherein the organic UV shielding agent comprises micronized methylene bis benzotriazolyl tetramethylbutylphenol.

12. A macroparticle powder in accordance with claim 11 wherein the organic UV shielding agent has an average particle size of less than 200 nm.

13. A macroparticle powder in accordance with claim 2 wherein said macroparticle powder comprises more than one ultraviolet shielding agent.

14. A macroparticle powder in accordance with claim 1 wherein the matrix material comprises agar.

15. A dispersion incorporating the macroparticle of claim 1.

16. A cosmetic incorporating the macroparticle of claim 1.

17. A sunscreen lotion incorporating the macroparticle of claim 1.

18. A method for forming a macroparticle powder capable of filtering ultraviolet radiation comprising:
(a) providing a feedstock comprising a UV shielding agent and a matrix material in a dispersing media;
(b) distributing the feed stock into a liquid material;
(c) forming said liquid material into droplets; and
(d) evaporating the dispersing media from the feed stock to produce a macroparticle powder comprising a plurality of macroparticle particles wherein the macroparticle particles comprise the UV shielding agent embedded in the matrix material.

19. The method of claim 18 wherein the dispersing media comprises water.

20. The method of claim 19 wherein the UV shielding agent comprises an inorganic UV filter selected from the group consisting of TiO₂, ZnO and combinations thereof.

21. The method of claim 20 wherein said metal oxide particles have an average particle size of from about 5 nm to about 50 nm.

22. The method of claim 18 wherein the UV shielding agent comprises a micronized organic UV shielding agent having an average particle size of less than 200 nm.

23. The method of claim 22 wherein the organic UV shielding agent comprises micronized Methylene Bis-Benzotriazolyl Tetramethylbutylphenol.

24. The method of claim 18, wherein said feedstock is provided by mixing a matrix solution with a sunscreen suspension, and wherein said of reparation and droplet formation is performed using a spray drying apparatus.

25. The method of claim 24, wherein said matrix solution as prepared by mixing agar with water.

26. A method of protecting human skin or human hair from ultraviolet radiation comprising treating said skin or hair with an effective protecting concentration of a composition comprising the product of the method of claim 24.

27. The method of claim 26 wherein the matrix material comprises agar.

28. A sunscreen personal care composition for protecting human skin or human hair from ultraviolet radiation which comprises an effective protecting concentration of a composition comprising macroparticle powder particles wherein the macroparticle powder particles comprise a UV shielding agent particle and a matrix material.

29. A sunscreen composition in accordance with claim 28 wherein the UV shielding agent comprises a UV shielding agent having an average particle size of less than 200 nm.
